(19) United States
(12) Patent Application Publication
(10) Pub. No.: US 2015/0291683 A1
(43) Pub. Date: Oct. 15, 2015

(54) MODIFIED APOL1 POLYPEPTIDES

(71) Applicants: Jason SAUNDERS, Kenilworth,, NJ (US); Michael HOHN, kenilworth,, NJ (US); Zhong LIU, Kenilworth,, NJ (US); MERCK SHARP & DOHME CORP., Rahway,, NJ (US)

(72) Inventors: Michael J. Hohn, Scotch Plains, NJ (US); Liu Zhong, Colts Neck, NJ (US); Jason Saunders, Somerset, NJ (US)

(73) Assignee: Merck Sharp & Dohme Corp., Rahway, NJ (US)

(21) Appl. No.: 14/443,257

(22) PCT Filed: Nov. 11, 2013
(86) PCT No.: PCT/US2013/069371

§ 371 Date: May 15, 2015

Related U.S. Application Data
(60) Provisional application No. 61/773,015, filed on Mar. 5, 2013.
(60) Provisional application No. 61/727,443, filed on Nov. 16, 2012.

(51) Int. Cl. C07K 14/775; C12P 21/02

(52) U.S. Cl. CPC C07K 14/775; C12P 21/02

(57) ABSTRACT

Modified ApoL1 polypeptides are provided, in particular, the modified polypeptides are expressed without an N-terminal or f-Methionine in bacterial cells. Also provided is a method of recombinantly producing the modified polypeptide.

### FIELD OF THE INVENTION

The present invention provides modified Apo-L1 proteins. In particular, it provides purified proteins, nucleic acids, and methods of recombinantly expressing these modified proteins.

### BACKGROUND OF THE INVENTION

The biosynthesis of proteins in genetically altered procaryotic cells results in expression of a protein having an N-formyl- or terminal methionine attached at the amino terminus. Since the addition of N-formylmethionine to the native protein may alter its biological activity, conformational stability, antigenicity, etc., it is most desirable, if possible, to remove it.

Protein synthesis in E. coli usually starts with the modified amino acid N-formylmethionine (fMet) which therefore is the N-terminal amino acid in most proteins. With few exceptions the N-formyl group is subsequently removed by peptide deformylase and, depending on the identity of the second amino acid, the N-terminal methionine may also be cleaved as well by methionine aminopeptidase (MAP). Especially in strongly over-expressed recombinant proteins fMet deformylation may become incomplete and since fMet is immunogenic, this can pose a problem for proteins of pharmaceutical interest.

Apolipoprotein L-I (ApoL1) is a human-specific serum protein that kills Trypanosoma brucei through ionic pore formation in endosomal membranes of the parasite. The T. brucei subspecies rhodesiense and gambiense resist this lytic activity and can infect humans, causing sleeping sickness. In the case of T. b. rhodesiense resistance to lysis involves interaction of the Serum Resistance-Associated (SRA) protein with the C-terminal helix of ApoL1. Normal human serum (NHS) is able to kill T. b. brucei, but not T. b. rhodesiense and T b. gambiense.

The human ApoL1 has, after processing of its secretion leader peptide, the N-terminal amino acid sequence Glu-Glu-Ala-Gly-. When over-expressed in E. coli, ApoL1 retains Met (and potentially residual fMet) at the N-terminus due to the large and charged glutamate at the second amino acid position.

In order to maintain high expression rates of ApoL1 in addition to complete f-Met removal, other techniques besides introducing peptidases into the host cell lines, are needed. The present invention fills this need by providing modified ApoL1 proteins that, when expressed, lack the terminal f-Met while maintaining a high expression level.

### BRIEF DESCRIPTION OF THE DRAWING

FIG. 1 shows the sequence analysis of the five N-terminal amino acids of: (FIG. 1A) ApoL1 (wt), expressed from pAVE012b, (FIG. 1B) ApoL1 (A2ins), expressed from pAVE012b, (FIG. 1C) ApoL1 (A2ins), expressed from pStaby1.2, (FIG. 1D) ApoL1 (EE2del), ApoL1 (EEA2del), expressed from pStaby1.2, and (FIG. 1E) ApoL1 (EEA2del), expressed from pStaby1.2. Amino acid sequences were identified by automated Edman degradation. Determined amino acid quantities, expressed in pmol, are shown for each reaction cycle, and the prevalent amino acid in each cycle is highlighted.

FIG. 2 shows RP-HPLC profiles of Endoproteinase Lys-C digested ApoL1 wild type (FIG. 2A), ApoL1 A2ins (FIG. 2B), and ApoL1 EE2del (FIG. 2C). The peak corresponding to the N-terminal peptide fragment is indicated. Shown amino acid sequences were derived from MS analysis. Determined masses were 2502.15 Da (predicted: 2502.54 Da) for ApoL1 wild type, 2442.15 Da (predicted: 2442.42 Da) for ApoL1 A2ins, and 2113.03 Da (predicted: 2113.13 Da) for ApoL1 EE2del (data not shown).

FIG. 3 shows growth inhibition of Trypanosomes in the presence of ApoL1. Trypanosoma brucei cell dilutions were incubated with the indicated amounts of ApoL1 in a final volume of 300 μl over a period of 20 hrs. at 37° C. Metabolic activity of the Trypanosomes was then assayed using the fluorescent redox indicator Alamar Blue.

### SUMMARY OF THE INVENTION

The present invention is based, in part, upon the discovery that post translational processing of wild type ApoL1 does not completely remove the terminal or f-Met.

The present invention encompasses a modified Apo-L1 polypeptide wherein the polypeptide lacks an N-terminal Methionine (Met) or formylmethionine when recombinantly expressed. In certain embodiments, the polypeptide comprises an amino acid residue inserted between the N-terminal Met and an adjacent residue, including an alanine residue inserted between the N-terminal Met and the adjacent residue. In a further embodiment, the adjacent residue is a glutamate residue. The present invention provides that the modified Apo-L1 polypeptide comprises the amino acid sequence of SEQ ID NO: 2 or SEQ ID NO: 4.

In another embodiment, the modified Apo-L1 polypeptide comprises a deletion of at least one amino acid residue adjacent to the N-terminal Met. In an alternative embodiment, the modified Apo-L1 comprises a deletion of at least two amino acid residues adjacent to the N-terminal Met, where the adjacent residues are glutamates.

The present invention provides that the modified ApoL1 polypeptide comprises the amino acid sequence of SEQ ID NO: 6. In an alternative embodiment, the modified Apo-L1 comprises a deletion of at least three amino acid residues adjacent to the N-terminal Met, where the adjacent residues are two glutamates and an alanine. The present invention provides that the modified ApoL1 polypeptide comprises the amino acid sequence of SEQ ID NO: 8. In certain embodiments, the modified ApoL1 polypeptide is recombinantly expressed by a bacterial cell, including an E. coli cell.

The present invention provides an isolated nucleic acid encoding the modified Apo-L1 polypeptide comprising the nucleic acid sequence selected from the group consisting of SEQ ID NOs: 1, 3, 5, and 7. Also provided is an expression vector comprising the nucleic acid selected from the group consisting of SEQ ID NOs: 1, 3, 5, and 7, and a host cell comprising the expression vector.

The present invention further provides a method of producing a polypeptide comprising: culturing the host cell in culture medium under conditions wherein the nucleic acid sequence is expressed, thereby producing the modified Apo-L1 polypeptide; and recovering the modified Apo-L1 polypeptide from the host cell or culture medium. In further embodiments, the host cell is a bacterial cell, including an E. coli cell.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

“Apolipoprotein L1” or “ApoL1” is meant a gene encoding human apolipoprotein L or the protein, ApoL1. ApoL1 is a secreted high density lipoprotein which binds to apolipoprotein A-I. Apolipoprotein A-I is a relatively abundant plasma protein and is the major apoprotein of HDL.

“Polypeptide fragment” is meant a portion of a polypeptide (e.g., an ApoL1 polypeptide, an anti-ApoL1 antibody, or an ApoL1 antagonist (e.g., an ApoL1-binding polypeptide, such as an SRA) or nucleic acid molecule encoding an ApoL1 polypeptide, an anti-ApoL1 antibody, an ApoL1 antagonist (e.g., an SRA), or an antisense ApoL1 molecule) having a region that is substantially identical to a portion of a reference protein or nucleic acid and retains at least 50% or 75%, more preferably 80%, 90%, or 95%, or even 99% of at least one biological activity of the reference protein or nucleic acid, but does not include the entire amino acid or nucleic acid sequence of the full-length polypeptide or nucleic acid molecule. For example, the fragment may have at least 1, at least 5, at least 10, 20, 30, 40, 50, 60, 70 80, 90, or 100, or more (e.g., up to 200, 300 or more) fewer amino acid residues or nucleic acid bases relative to the full-length polypeptide or nucleic acid molecule (e.g., an ApoL1 polypeptide, an anti-ApoL1 antibody, or an ApoL1 antagonist (e.g., an SRA) or nucleic acid molecule encoding an ApoL1 polypeptide, an anti-ApoL1 antibody, an ApoL1 antagonist (e.g., an SRA), or an antisense APOL1 molecule).

By “isolated” (e.g., as an “isolated” biological component, such as a nucleic acid molecule, protein, antibody, or cell, or as an “isolated” chemical component, such as a compound or other chemical therapeutic agent) is meant that a component has been substantially separated or purified away from other biological (or chemical) components, e.g., as in the cell of an organism in which the component naturally occurs. For example, a component is “isolated” if it is enriched in a composition, relative to other components in the composition, such that it constitutes at least 30%, more preferably at least 50%, or even more preferably at least 75% or more of the composition. A component is “substantially isolated” or “substantially purified” if it is enriched, relative to other components in a composition, such that it constitutes at least 85%, more preferably at least 90%, or even more preferably 95%, 97%, or 99% or more of the composition. Nucleic acids and proteins that have been “isolated” include, e.g., nucleic acid molecules that encode an ApoL1 polypeptide or fragment thereof and nucleic acid therapeutics of the invention, ApoL1 polypeptides or fragments thereof, each of which may be purified by standard purification methods. The term also embraces nucleic acid molecules and proteins prepared by recombinant expression in a host cell as well as chemically synthesized nucleic acid molecules, peptides, and polypeptides (e.g., one or more of the nucleic acid molecules, proteins, and antibodies of the invention).

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; an promoter or enhancer is operably linked to a coding sequence if it affects the transcription of the sequence; or a ribosome binding site is operably linked to a coding sequence if it is positioned so as to facilitate translation.

Generally, “operably linked” means that the DNA sequences being linked are contiguous, and, in the case of a secretory leader, contiguous and in reading phase. However, enhancers do not have to be contiguous Linking is accomplished by ligation at convenient restriction sites. If such sites do not exist, the synthetic oligonucleotide adaptors or linkers are used in accordance with conventional practice.

The term “epitope tagged” when used herein refers to a chimeric polypeptide comprising modified ApoL1 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

### II. General

Apolipoprotein L1 (ApoL1) is a human-specific serum apolipoprotein bound to high-density lipoprotein (HDL). Although its complete function is not yet fully understood, it is assumed that ApoL1 plays a major role in the defense against blood-borne parasites like trypanosomes. In addition, recent findings revealed a correlation between two coding sequence variants of ApoL1 and the increased occurrence of nondiabetic chronic kidney disease in African Americans compared to European Americans. ApoL1 therefore is a highly interesting protein for pharmaceutical research.

In its native form, human ApoL1 is expressed with an N-terminal export leader peptide (MEGAALLRVSVLCIWMSALFLGVGVRA; SEQ ID NO: 13) which is cleaved upon protein secretion from the cell, so that the N-terminus of the mature protein starts with EEAGA (SEQ ID NO: 14). For recombinant expression in E. coli, this eukaryotic signal peptide needs to be removed and a methionine encoding ATG start codon is added in its place to the 5′ end of the coding sequence. In E. coli, protein expression usually is initiated with the modified amino acid formyl-methionine (fMet). In most cases, the formyl group is post-translationally removed from the methionine moiety by peptide deformylase. Depending on the nature of the adjacent amino acid, the N-terminal Met is then cleaved off by methionine amino peptidase (MAP).

N-terminal protein sequencing showed that wild type ApoL1 over-expressed in E. coli retains the N-terminal Met and possibly residual fMet. This poses a problem for downstream applications since fMet is recognized as foreign by the human immune system. It has been reported earlier that cleavage of the N-terminal Met by MAP is very inefficient when the amino acid following the initiator-Met is a glutamate. To solve this problem, ApoL1 constructs were made where either an alanine has been inserted immediately downstream of the initiator-Met, or where the first two (Glu-Glu) or three (Glu-Glu-Ala) amino acid residues following the initiator-Met in the amino acid sequence of ApoL1 were deleted. These protein constructs were expressed in E. coli, with and without C-terminal His6-tag. Purified ApoL1 proteins were analyzed by N-terminal peptide sequencing and tandem mass spectrometry.

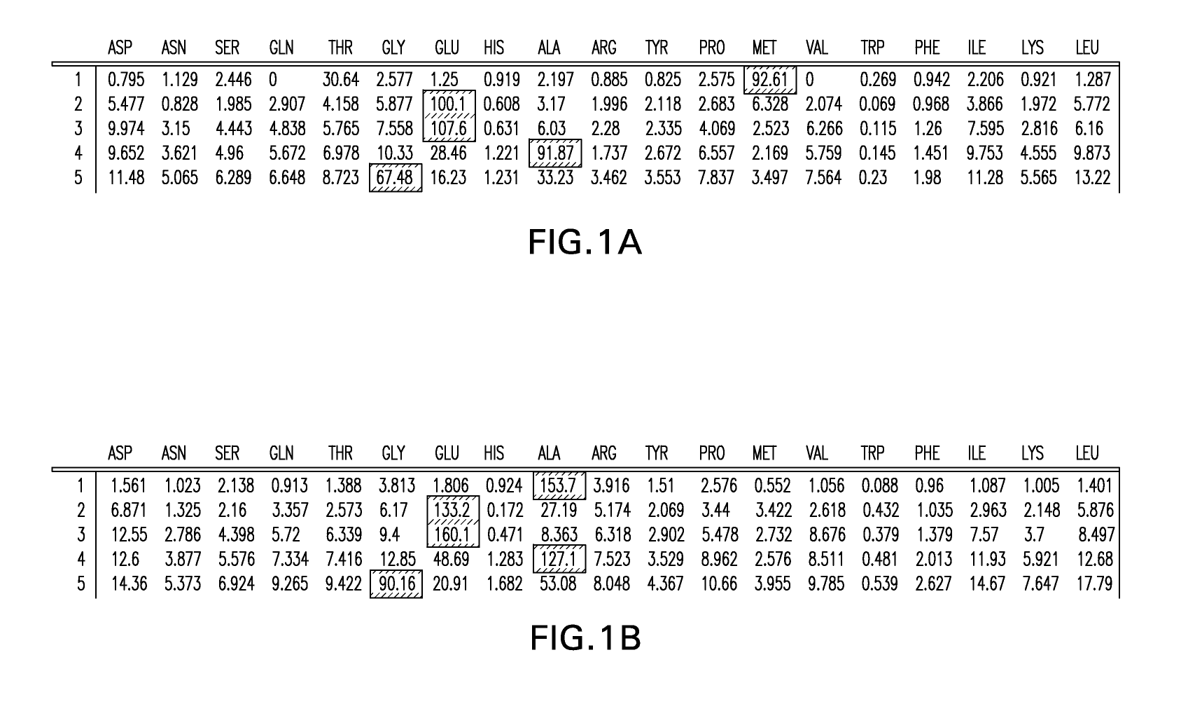

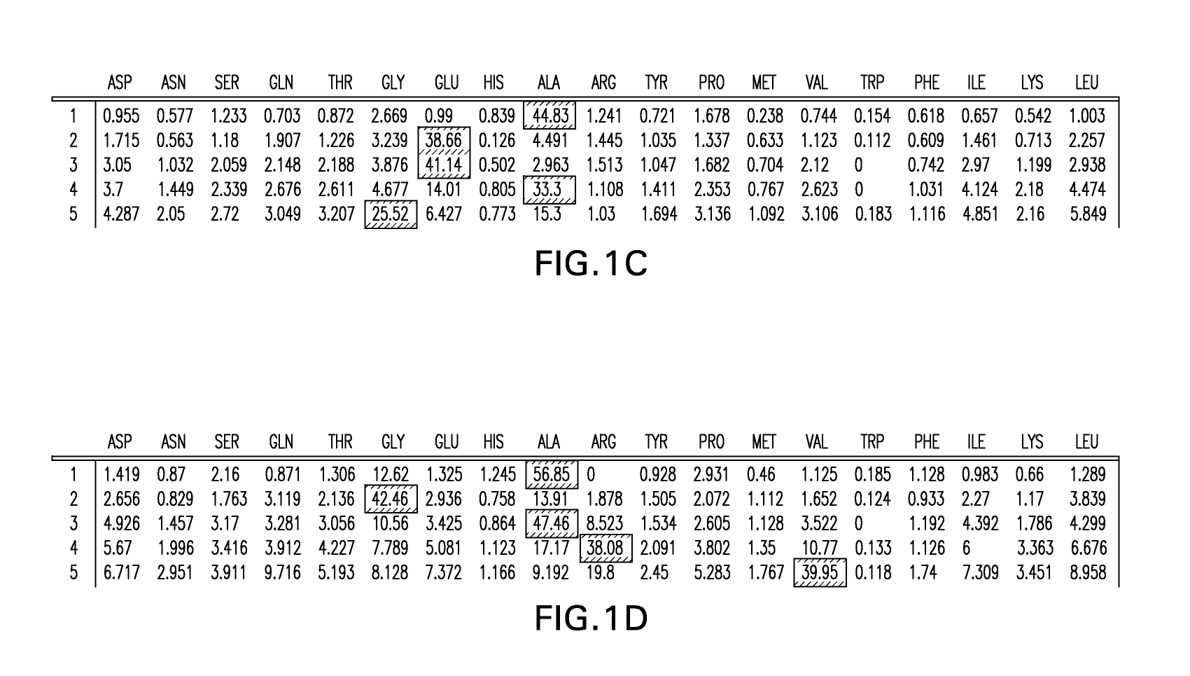

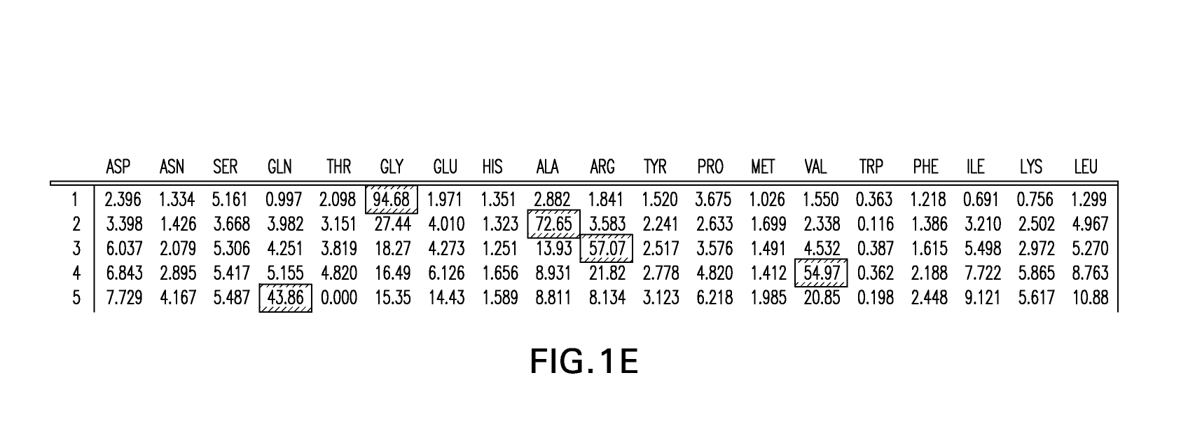

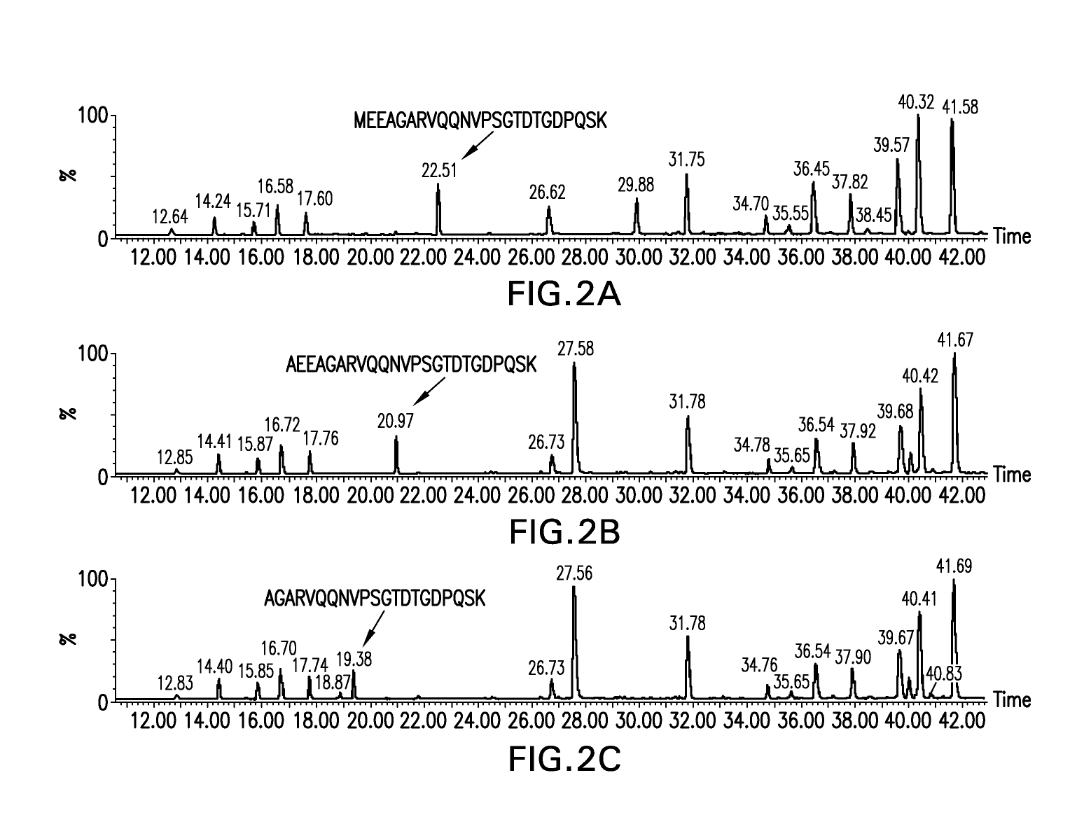

Results from both N-terminal sequencing (FIG. 1) and peptide mapping (FIG. 2) show that either the genetic insertion of an alanine immediately downstream of the initiator Met, or the deletion of the first two glutamic acid residues in the amino acid sequence of ApoL1 result in quantitative removal of the initiator Met in vivo in E. coli.

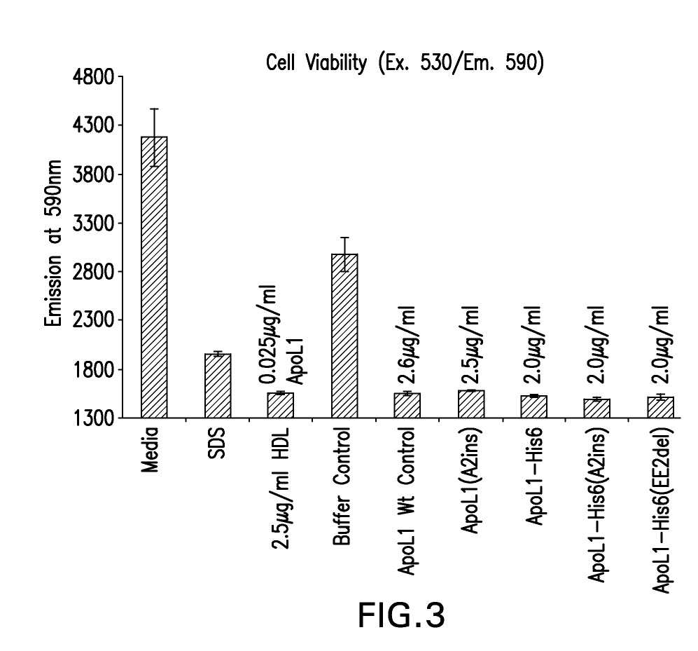

Trypanosome growth inhibition assays using wild type or modified ApoL1 proteins were carried out and demonstrate that neither the above described alterations of the ApoL1 N-terminus, nor the addition of a C-terminal His-tag affect the trypanolytic activity of ApoL1 (see, e.g., FIG. 3).

### Polypeptides

The present invention provides modified ApoL1 polypeptides, in particular polypeptides that lack the N-terminal or formyl-Methionine. Modified ApoL1 polypeptides can be prepared by introducing appropriate nucleotide changes into the ApoL1 DNA, and/or by synthesis of the desired modified Apo-L1 polypeptide. Those skilled in the art will appreciate that amino acid changes may alter post-translational processes of the modified ApoL1 polypeptide, such as changing the number or position of glycosylation sites or altering the membrane anchoring characteristics.

Variations in the native full-length sequence ApoL1 polypeptide or in various domains of the ApoL1 polypeptide described herein, can be made, for example, using any of the techniques and guidelines for conservative and non-conservative mutations set forth, for instance, in U.S. Pat. No. 5,364,934. Variations may be a substitution, deletion or insertion of one or more codons encoding ApoL1 that results in a change in the amino acid sequence of the modified ApoL1 polypeptide as compared with the native sequence ApoL1 polypeptide. Optionally the variation is by substitution of at least one amino acid with any other amino acid in one or more of the domains of the modified ApoL1 polypeptide. Guidance in determining which amino acid residue may be inserted, substituted or deleted without adversely affecting the desired activity may be found by comparing the sequence of the modified ApoL1 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Modified ApoL1 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the modified ApoL1 polypeptide.

Modified ApoL1 polypeptide fragments may be prepared by any of a number of conventional techniques. Desired peptide fragments may be chemically synthesized. An alternative approach involves generating modified ApoL1 polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR.

Preferably, modified ApoL1 polypeptide fragments share at least one biological and/or immunological activity with the native modified ApoL1 polypeptide disclosed herein.

In particular embodiments, conservative substitutions of interest are shown in Table 2 under the heading of preferred substitutions. If such substitutions result in a change in biological activity, then more substantial changes, denominated exemplary substitutions in Table 2, or as further described below in reference to amino acid classes, are introduced and the products screened.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

### Nucleic Acids

The present invention further comprises the nucleic acids encoding the modified ApoL1 polypeptide and antigen binding fragments thereof disclosed herein. For example, the present invention includes the nucleic acids (SEQ ID NOs: 1, 3, 5, and 7) as well as nucleic acids which hybridize thereto.

In general, the nucleic acids hybridize under low, moderate or high stringency conditions, and encode modified ApoL1 polypeptides. A first nucleic acid molecule is “hybridizable” to a second nucleic acid molecule when a single stranded form of the first nucleic acid molecule can anneal to the second nucleic acid molecule under the appropriate conditions of temperature and solution ionic strength (see Sambrook, et al., supra). The conditions of temperature and ionic strength determine the “stringency” of the hybridization. Typical low stringency hybridization conditions include 55° C., 5×SSC, 0.1% SDS and no formamide; or 30% formamide, 5×SSC, 0.5% SDS at 42° C. Typical moderate stringency hybridization conditions are 40% formamide, with 5× or 6×SSC and 0.1% SDS at 42° C. High stringency hybridization conditions are 50% formamide, 5× or 6×SSC at 42° C. or, optionally, at a higher temperature (e.g., 57° C., 59° C., 60° C., 62° C., 63° C., 65° C. or 68° C.). In general, SSC is 0.15M NaCl and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, e.g., oligonucleotides, the position of mismatches becomes more important, and the length of the oligonucleotide determines its specificity (see Sambrook, et al., supra, 11.7-11.8).

Also included in the present invention are modified ApoL1 polypeptides comprising amino acid sequences that are at least about 70% identical, preferably at least about 80% identical, more preferably at least about 90% identical and most preferably at least about 95% identical (e.g., 95%, 96%, 97%, 98%, 99%, 100%) to any of the ApoL1 reference sequences provided herein when the comparison is performed by a BLAST algorithm wherein the parameters of the algorithm are selected to give the largest match between the respective sequences over the entire length of the respective reference sequences. Modified ApoL1 polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference amino acid sequences when the comparison is performed with a BLAST algorithm wherein the parameters of the algorithm are selected to give the largest match between the respective sequences over the entire length of the respective reference sequences, are also included in the present invention.

Sequence identity refers to the degree to which the amino acids of two polypeptides are the same at equivalent positions when the two sequences are optimally aligned. Sequence similarity includes identical residues and nonidentical, biochemically related amino acids. Biochemically related amino acids that share similar properties and may be interchangeable are discussed above.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

The present invention also provides expression vectors comprising the isolated nucleic acids of the invention, wherein the nucleic acid is operably linked to control sequences that are recognized by a host cell when the host cell is transfected with the vector. Also provided are host cells comprising an expression vector of the present invention and methods for producing the modified polypeptide thereof disclosed herein comprising culturing a host cell harboring an expression vector encoding the modified polypeptide in culture medium, and isolating the modified polypeptide from the host cell or culture medium.

### Method of Making Modified ApoL1 Polypeptides

The modified ApoL1 polypeptides disclosed herein may also be produced recombinantly (e.g., in an E. coli/T7 expression system as discussed above). In this embodiment, nucleic acids encoding the polypeptides of the invention may be inserted into a pET-based plasmid and expressed in the E. coli/T7 system. There are several methods by which to produce recombinant polypeptides which are known in the art. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W31 10 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting.

The broad scope of this invention is best understood with reference to the following examples, which are not intended to limit the inventions to the specific embodiments.

### Examples

### I. General Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification is described (Cutler (ed.) (2004) Protein Purification Protocols, 2^{nd} Ed. Humana Press, Totowa, N.J.; and Scopes (1994) Protein Purification: Principles and Practice, Springer-Verlag, NY, NY).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### II. Vector Subcloning

Cloning of ApoL1 gene variants into pAVE012B. Genes (including stop codon) encoding wild type ApoL1 and ApoL1 (A2ins—with an Ala insertion at amino acid position 2) were codon-optimized for E. coli and synthesized by GenScript. An N-terminal NdeI restriction site and a C-terminal XhoI restriction site were added for subcloning. The genes were ligated into NdeI/XhoI-digested plasmid pAVE012b (Fujifilm Diosynth Biotechnologies), resulting in pAVE012b-ApoL1WT and pAVE012b-ApoL1 (A2ins), respectively.

Cloning of ApoL1 gene variants into pStaby1.2 Genes (without stop codon) encoding wild type ApoL1 (SEQ ID NOs: 9 or 11), ApoL1 (A2ins; SEQ ID NOs: 1 or 3), ApoL1 (EE2del—with the glutamates at amino acid positions 2 and 3 are deleted; SEQ ID NO: 5), and ApoL1 (EEA2del—with the glutamates at amino acid positions 2 and 3, and the alanine at amino acid position 4 deleted; SEQ ID NO: 7) were codon-optimized for E. coli and synthesized by GenScript. An N-terminal NdeI restriction site and a C-terminal XhoI restriction site were added for subcloning. The genes were ligated into Plasmid pStaby1.2 (Delphi Genetics) using restriction sites for NdeI and XhoI. By this method, a His6-tag was added to the C-terminus of the encoded proteins. The resulting plasmids were pStaby1.2-ApoL1 (A2ins), and pStaby1.2-ApoL1 (EE2del).

### III. Protein Expression

ApoL1 expression from pAVE012B. E. coli BL21 (DE3) cells (Novagen), transformed with pAVE012b constructs pAVE012b-ApoL1WT, or pAVE012b-ApoL1 (A2ins), respectively, were grown in 400 mL Auto-Induction Medium (1.2% tryptone, 2.4% yeast extract, 0.05% D-(+)-glucose, 0.5% glycerol, 0.2% D-lactose, 25 mM (NH₄)2SO₄, 50 mM Na₂HPO₄, 50 mM KH₂PO₄, 1 mM MgSO₄, 1× Vitamin Mix (1), and 0.003% SAG-anti foam) for 12 h at 42° C. with agitation (350 rpm). Protein expression was induced by the lactose auto-induction method according to (1). Grown cells were harvested by centrifugation and cell pellets were stored at −80° C.

ApoL1 expression from pStaby1.2. E. coli SE1 cells (Delphi Genetics) were transformed with pStaby1.2 constructs pStaby1.2-ApoL1 (A2ins), pStaby1.2-ApoL1 (EE2del), and ApoL1 (EEA2del), respectively. Protein expression was induced by auto-induction under the same conditions as described above. Harvested cells were stored at −80° C.

### IV. Protein Purification

ApoL1 purification by Hydrophobic Interaction Chromatography (HIC). 50 g frozen cell pellet was resuspended in 750 ml Lysis Buffer (50 mM Tris/HCl, pH 8.3; 5 mM EDTA) using a PT 6100 homogenizer (Kinematica). Cells were broken by four passages through a Microfluidizer model 110F (Microfluidics). Inclusion bodies were then purified from the cell lysate by centrifugation. After washing in Wash Buffer 1 (50 mM Tris/HCl, pH 8.4; 100 mM NaCl; 1M urea), and Wash Buffer 2 (50 mM Tris/HCl, pH 8.2; 100 mM NaCl), 0.8-1 g inclusion body pellet was dissolved in 25 ml Dissolving Buffer.

Purification of His6-tagged ApoL1 constructs by Ni2+-NTA affinity chromatography under denaturing conditions. Approximately 1.2 g of frozen cell pellets were resuspended in 8 ml Lysis Buffer (lx PBS (Boston BioProducts); lx BugBuster (Millipore); 125U Benzonase (Millipore); 5 KU Lysozyme (Sigma)). Cell lysis occurred during a 20 min incubation step at RT. Insoluble inclusion bodies were recovered by centrifugation, and washed 3 times in 7 ml Wash Buffer (lx PBS; lx BugBuster; 5 KU Lysozyme). Inclusion body pellets were resuspended over night in 40 ml Buffer A (100 mM KH2PO4; 10 mM Tris/HCl; 8 M Urea; adjusted to pH 8.0).

His6-tagged protein was further purified by Ni2+-NTA agarose (Qiagen) affinity chromatography. Column-bound protein was washed with 6 ml Buffer B (100 mM KH2PO4; 10 mM Tris/HCl; 8 M Urea; adjusted to pH 6.3), and was eluted with 1 ml Buffer C (100 mM KH2PO4; 10 mM Tris/HCl; 8 M Urea; adjusted to pH 4.5). Purity (>95%) of eluted ApoL1-His6 fractions was confirmed by SDS-PAGE.

### V. Protein Analysis

N-terminal protein sequencing. Purified ApoL1 proteins were subjected to SDS-PAGE and electro-blotted to a PVDF membrane. Membranes were then transferred to the Tufts University Core Facility (Tufts Medical School, Boston, Mass.), where proteins were N-terminally sequenced by automated Edman Degradation using ABI 494 Protein Sequencers.

Peptide mapping. 50 μg samples were dissolved into reducing buffer to a final volume of 100 μl containing 50 mM Tris buffer at pH 8.0, 6 M Guanidine HCl, 5 mM EDTA, 20 mM DTT and incubated at 56° C. for 30 min. The sample were cooled at room temperature for 5 min and subsequently alkylated with 50 mM iodoacetamide at room temperature for 30 min in the dark. The alkylation reaction was terminated by adding 1 μL of 1 M DTT.

For Lys-C digestion, reduced and alkylated samples were diluted with digest buffer (50 mM Tris buffer at pH 8.0, 5 mM EDTA) to a final volume of 600 μL and treated with an aliquot of Lys-C (Wako) at a ratio of 1:40 (enzyme/substrate weight ratio). The mixture was incubated at 37° C. for 1 hour, and another aliquot of Lys-C at a ratio of 1:40 was added and incubated for another 3 hours at 37° C. The reaction was terminated by adding 15 μL of 20% TFA.

Resulting peptide fragments were separated using reverse phase high performance liquid chromatography (RP-HPLC) with on-line UV and MS detection. Peptide peaks were annotated based on their predicted masses in combination with MS/MS sequencing information.

### VI. Trypanosome Growth Inhibition Assays

Equal concentrations of Trypanosoma brucei cells were mixed with 2.0-2.5 μg/ml (final concentrations) wild type ApoL1, ApoL1 (A2ins), ApoL1-His6, ApoL1-His6 (A2ins), or ApoL1-His6 (EE2del), respectively, in a final volume of 300 μl Trypanosome Culture Media. 2.5 μg/ml HDL, 0.13% SDS, or 4 μl ApoL1-buffer (20 mM HEPES, 200 mM NaCl, 10% glycerol; pH 7.3) were used as controls. Cells were incubated for 20 hrs. at 37° C. in the presence of 5% CO₂. To assay cell viability, 20 μl Alamar Blue reagent (Invitrogen) was added to the cell suspensions and sample plates were incubated for further 4 hrs. at 37° C. Fluorescence was then determined using a Labsystems Fluoroskan II plate reader (excitation: 544 nm, emission: 590 nm).

DESCRIPTION OF SEQUENCE LISTING

| SEQ ID NO | Description |
|---|---|
| 1 | ApoL1 (A2ins) nucleic acid |
| 2 | ApoL1 (A2ins) amino acid |
| 3 | ApoL1-His6 (A2ins) nucleic acid |
| 4 | ApoL1-His6 (A2ins) amino acid |
| 5 | ApoL1-His6 (EE2del) nucleic acid |
| 6 | ApoL1-His6 (EE2del) amino acid |
| 7 | ApoL1-His6 (EEA2del) nucleic acid |
| 8 | ApoL1-His6 (EEA2del) amino acid |
| 9 | ApoL1 (WT) nucleic acid |
| 10 | ApoL1 (WT) amino acid |
| 11 | ApoL1-His6 (WT) nucleic acid |
| 12 | ApoL1-His6 (WT) amino acid |
| 13 | ApoL1 N-terminal export leader peptide |
| 14 | ApoL1 mature polypeptide N-terminus |

What is claimed is:

1. A modified Apo-L1 polypeptide wherein the polypeptide lacks an N-terminal Methionine (Met) or formylmethionine when recombinantly expressed.

2. The modified Apo-L1 polypeptide of claim 1 wherein the polypeptide comprises an amino acid residue inserted between the N-terminal Met and an adjacent residue.

3. The modified Apo-L1 polypeptide of claim 2 comprising an alanine residue inserted between the N-terminal Met and the adjacent residue.

4. The modified Apo-L1 polypeptide of claim 3, wherein the adjacent residue is a glutamate residue.

5. The modified Apo-L1 polypeptide of claim 1 comprising the amino acid sequence of SEQ ID NO: 2.

6. The modified Apo-L1 polypeptide of claim 1 comprising the amino acid sequence of SEQ ID NO: 4.

7. The modified Apo-L1 polypeptide of claim 1 wherein the polypeptide comprises a deletion of at least one amino acid residue adjacent to the N-terminal Met.

8. The modified Apo-L1 polypeptide of claim 1, wherein the polypeptide comprises a deletion of at least two amino acid residues adjacent to the N-terminal Met.

9. The modified Apo-L1 polypeptide of claim 8 wherein the adjacent residues are glutamates.

10. The modified polypeptide of claim 1, comprising the amino acid sequence of SEQ ID NO: 6.

11. The modified Apo-L1 polypeptide of claim 1, wherein the polypeptide comprises a deletion of at least three amino acid residues adjacent to the N-terminal Met.

12. The modified Apo-L1 polypeptide of claim 11 wherein the adjacent residues are two glutamates and one alanine.

13. The modified polypeptide of claim 1, comprising the amino acid sequence of SEQ ID NO: 8.

14. The modified polypeptide of claim 1 wherein the polypeptide is recombinantly expressed by a bacterial cell.

15. The modified polypeptide of claim 14, wherein the bacterial cell is an E. coli cell.

16. An isolated nucleic acid encoding the modified Apo-L1 polypeptide of claim 1, wherein the nucleic acid is selected from the group consisting of SEQ ID NOs: 1, 3, 5 and 7.

17. An expression vector comprising the nucleic acid of claim 16.

18. A host cell comprising the expression vector of claim 17.

19. A method of producing a polypeptide comprising:
a) ring the host cell of claim 18 in culture medium under conditions wherein the nucleic acid sequence is expressed, thereby producing the modified Apo-L1 polypeptide; and
b) recovering the modified Apo-L1 polypeptide from the host cell or culture medium.

20. The method of claim 19, wherein the host cell is a bacterial cell.

21. The bacterial cell of claim 20, wherein the bacterial cell is an E. coli cell.


SEQUENCE LISTING (12 sequences)

SEQ ID NO: 1 - DNA, 1122 bp, Homo sapiens
atggcggaagaagctggcgcgcgtgttcagcaaaatgtcccgtcgggtaccgatacgggtgacccgcagagcaaaccgctgggtgattgggcagcaggtaccatggacccggaaagctctatcttcatcgaagatgccatcaaatacttcaaagaaaaagtgtctacgcaaaacctgctgctgctgctgaccgacaatgaagcctggaacggctttgttgcagctgcggaactgccgcgtaatgaagcagatgaactgcgcaaagccctggacaacctggcacgtcagatgatcatgaaagataaaaactggcatgacaagggtcagcaataccgtaactggtttctgaaagaattcccgcgcctgaaaagcaaactggaagataatattcgtcgcctgcgtgcactggcagacggtgttcagaaagtccacaaaggtaccacgatcgcgaacgtggttagcggctctctgagtattagttccggtatcctgaccctggtgggcatgggtctggcaccgttcaccgaaggcggttccctggttctgctggaaccgggcatggaactgggtattacggccgcactgaccggcattacgtcatcgaccatcgattatggtaaaaaatggtggacccaggctcaagcgcatgatctggtcattaaaagtctggacaaactgaaagaagtgaaagaatttctgggcgaaaatatctctaacttcctgagtctggctggtaatacgtaccagctgacccgcggcattggtaaagatatccgtgcactgcgtcgcgcccgcgcaaacctgcaaagcgttccgcacgcttccgcgtcacgtccgcgcgtcacggaaccgatttcggcagaaagcggcgaacaggtggaacgtgttaatgaaccgtccatcctggaaatgtcacgcggtgtgaaactgaccgatgtcgcgcctgtgagctttttcctggtgctggacgtcgtgtatctggtttacgaatctaaacatctgcacgaaggtgctaaaagtgaaaccgcggaagaactgaaaaaagttgcccaggaactggaagaaaaactgaacatcctgaacaacaactacaaaatcctgcaggcagatcaagaactgtga

SEQ ID NO: 2 - protein, 373 aa, Homo sapiens
MAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 3 - DNA, 1146 bp, Homo sapiens
atggcggaagaagctggcgcgcgtgttcagcaaaatgtcccgtcgggtaccgatacgggtgacccgcagagcaaaccgctgggtgattgggcagcaggtaccatggacccggaaagctctatcttcatcgaagatgccatcaaatacttcaaagaaaaagtgtctacgcaaaacctgctgctgctgctgaccgacaatgaagcctggaacggctttgttgcagctgcggaactgccgcgtaatgaagcagatgaactgcgcaaagccctggacaacctggcacgtcagatgatcatgaaagataaaaactggcatgacaagggtcagcaataccgtaactggtttctgaaagaattcccgcgcctgaaaagcaaactggaagataatattcgtcgcctgcgtgcactggcagacggtgttcagaaagtccacaaaggtaccacgatcgcgaacgtggttagcggctctctgagtattagttccggtatcctgaccctggtgggcatgggtctggcaccgttcaccgaaggcggttccctggttctgctggaaccgggcatggaactgggtattacggccgcactgaccggcattacgtcatcgaccatcgattatggtaaaaaatggtggacccaggctcaagcgcatgatctggtcattaaaagtctggacaaactgaaagaagtgaaagaatttctgggcgaaaatatctctaacttcctgagtctggctggtaatacgtaccagctgacccgcggcattggtaaagatatccgtgcactgcgtcgcgcccgcgcaaacctgcaaagcgttccgcacgcttccgcgtcacgtccgcgcgtcacggaaccgatttcggcagaaagcggcgaacaggtggaacgtgttaatgaaccgtccatcctggaaatgtcacgcggtgtgaaactgaccgatgtcgcgcctgtgagctttttcctggtgctggacgtcgtgtatctggtttacgaatctaaacatctgcacgaaggtgctaaaagtgaaaccgcggaagaactgaaaaaagttgcccaggaactggaagaaaaactgaacatcctgaacaacaactacaaaatcctgcaggcagatcaagaactgctcgagcaccaccaccaccaccactga

SEQ ID NO: 4 - protein, 381 aa, Homo sapiens
MAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQELLEHHHHHH

SEQ ID NO: 5 - DNA, 1137 bp, Homo sapiens
atggctggcgcgcgtgttcagcaaaatgtcccgtcgggtaccgatacgggtgacccgcagagcaaaccgctgggtgattgggcagcaggtaccatggacccggaaagctctatcttcatcgaagatgccatcaaatacttcaaagaaaaagtgtctacgcaaaacctgctgctgctgctgaccgacaatgaagcctggaacggctttgttgcagctgcggaactgccgcgtaatgaagcagatgaactgcgcaaagccctggacaacctggcacgtcagatgatcatgaaagataaaaactggcatgacaagggtcagcaataccgtaactggtttctgaaagaattcccgcgcctgaaaagcaaactggaagataatattcgtcgcctgcgtgcactggcagacggtgttcagaaagtccacaaaggtaccacgatcgcgaacgtggttagcggctctctgagtattagttccggtatcctgaccctggtgggcatgggtctggcaccgttcaccgaaggcggttccctggttctgctggaaccgggcatggaactgggtattacggccgcactgaccggcattacgtcatcgaccatcgattatggtaaaaaatggtggacccaggctcaagcgcatgatctggtcattaaaagtctggacaaactgaaagaagtgaaagaatttctgggcgaaaatatctctaacttcctgagtctggctggtaatacgtaccagctgacccgcggcattggtaaagatatccgtgcactgcgtcgcgcccgcgcaaacctgcaaagcgttccgcacgcttccgcgtcacgtccgcgcgtcacggaaccgatttcggcagaaagcggcgaacaggtggaacgtgttaatgaaccgtccatcctggaaatgtcacgcggtgtgaaactgaccgatgtcgcgcctgtgagctttttcctggtgctggacgtcgtgtatctggtttacgaatctaaacatctgcacgaaggtgctaaaagtgaaaccgcggaagaactgaaaaaagttgcccaggaactggaagaaaaactgaacatcctgaacaacaactacaaaatcctgcaggcagatcaagaactgctcgagcaccaccaccaccaccactga

SEQ ID NO: 6 - protein, 378 aa, Homo sapiens
MAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQELLEHHHHHH

SEQ ID NO: 7 - DNA, 1119 bp, Homo sapiens
atggaagaagctggcgcgcgtgttcagcaaaatgtcccgtcgggtaccgatacgggtgacccgcagagcaaaccgctgggtgattgggcagcaggtaccatggacccggaaagctctatcttcatcgaagatgccatcaaatacttcaaagaaaaagtgtctacgcaaaacctgctgctgctgctgaccgacaatgaagcctggaacggctttgttgcagctgcggaactgccgcgtaatgaagcagatgaactgcgcaaagccctggacaacctggcacgtcagatgatcatgaaagataaaaactggcatgacaagggtcagcaataccgtaactggtttctgaaagaattcccgcgcctgaaaagcaaactggaagataatattcgtcgcctgcgtgcactggcagacggtgttcagaaagtccacaaaggtaccacgatcgcgaacgtggttagcggctctctgagtattagttccggtatcctgaccctggtgggcatgggtctggcaccgttcaccgaaggcggttccctggttctgctggaaccgggcatggaactgggtattacggccgcactgaccggcattacgtcatcgaccatcgattatggtaaaaaatggtggacccaggctcaagcgcatgatctggtcattaaaagtctggacaaactgaaagaagtgaaagaatttctgggcgaaaatatctctaacttcctgagtctggctggtaatacgtaccagctgacccgcggcattggtaaagatatccgtgcactgcgtcgcgcccgcgcaaacctgcaaagcgttccgcacgcttccgcgtcacgtccgcgcgtcacggaaccgatttcggcagaaagcggcgaacaggtggaacgtgttaatgaaccgtccatcctggaaatgtcacgcggtgtgaaactgaccgatgtcgcgcctgtgagctttttcctggtgctggacgtcgtgtatctggtttacgaatctaaacatctgcacgaaggtgctaaaagtgaaaccgcggaagaactgaaaaaagttgcccaggaactggaagaaaaactgaacatcctgaacaacaactacaaaatcctgcaggcagatcaagaactgtga

SEQ ID NO: 8 - protein, 372 aa, Homo sapiens
MEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 9 - DNA, 1143 bp, Homo sapiens
atggaagaagctggcgcgcgtgttcagcaaaatgtcccgtcgggtaccgatacgggtgacccgcagagcaaaccgctgggtgattgggcagcaggtaccatggacccggaaagctctatcttcatcgaagatgccatcaaatacttcaaagaaaaagtgtctacgcaaaacctgctgctgctgctgaccgacaatgaagcctggaacggctttgttgcagctgcggaactgccgcgtaatgaagcagatgaactgcgcaaagccctggacaacctggcacgtcagatgatcatgaaagataaaaactggcatgacaagggtcagcaataccgtaactggtttctgaaagaattcccgcgcctgaaaagcaaactggaagataatattcgtcgcctgcgtgcactggcagacggtgttcagaaagtccacaaaggtaccacgatcgcgaacgtggttagcggctctctgagtattagttccggtatcctgaccctggtgggcatgggtctggcaccgttcaccgaaggcggttccctggttctgctggaaccgggcatggaactgggtattacggccgcactgaccggcattacgtcatcgaccatcgattatggtaaaaaatggtggacccaggctcaagcgcatgatctggtcattaaaagtctggacaaactgaaagaagtgaaagaatttctgggcgaaaatatctctaacttcctgagtctggctggtaatacgtaccagctgacccgcggcattggtaaagatatccgtgcactgcgtcgcgcccgcgcaaacctgcaaagcgttccgcacgcttccgcgtcacgtccgcgcgtcacggaaccgatttcggcagaaagcggcgaacaggtggaacgtgttaatgaaccgtccatcctggaaatgtcacgcggtgtgaaactgaccgatgtcgcgcctgtgagctttttcctggtgctggacgtcgtgtatctggtttacgaatctaaacatctgcacgaaggtgctaaaagtgaaaccgcggaagaactgaaaaaagttgcccaggaactggaagaaaaactgaacatcctgaacaacaactacaaaatcctgcaggcagatcaagaactgctcgagcaccaccaccaccaccactga

SEQ ID NO: 10 - protein, 380 aa, Homo sapiens
MEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQELLEHHHHHH

SEQ ID NO: 11 - protein, 27 aa, Homo sapiens
MEGAALLRVSVLCIWMSALFLGVGVRA

SEQ ID NO: 12 - protein, 5 aa, Homo sapiens
EEAGA